(19) United States
(12) Patent Application Publication
(10) Pub. No.: US 2016/0368983 A1
(43) Pub. Date: Dec. 22, 2016

(54) COMPOSITIONS AND METHODS FOR TREATING OBESITY

(71) Applicants: Kim D. JANDA, La Jolla, CA (US); THE SCRIPPS RESEARCH INSTITUTE, La Jolla, CA (US)

(72) Inventor: Kim D. Janda, La Jolla, CA (US)

(21) Appl. No.: 14/901,799

(22) PCT Filed: Jul. 1, 2014
(86) PCT No.: PCT/US14/45023

§ 371 Date: Dec. 29, 2015

Related U.S. Application Data
(60) Provisional application No. 61/842,078, filed on Jul. 2, 2013.

(51) Int. Cl. C07K 16/26

(52) U.S. Cl. CPC C07K 16/26; C07K 2317/622; C07K 2317/55; C07K 2317/54; A61K 2039/505

(57) ABSTRACT

The present invention provides anti-ghrelin antibodies or antigen-binding molecules that are capable of sequestering ghrelin and inhibiting ghrelin-mediated cellular activities. Also provided in the invention are therapeutic applications of combinations of these antibodies, e.g., to treat or prevent obesity.

### BACKGROUND OF THE INVENTION

Obesity is a chronic, costly, and globally prevalent condition, with excess caloric intake a suspected etiologic factor. Approximately 1 billion people worldwide are overweight or obese (body mass index=25-29.9 or >30 kg/m², respectively). These conditions are associated with significant morbidity and mortality and for which new treatments are needed. Nonsurgical treatments of obesity are modestly efficacious, and weight loss maintenance is hampered by anti-famine homeostatic mechanisms.

Human ghrelin is a 28-amino acid acylated peptide (Kojima et al., Nature. 402:656-60, 1999). It is released mainly from endocrine cells of the stomach and upper gastrointestinal tract but also expressed in testes, kidney, pituitary, pancreas, lymphocytes, and brain. Gastric ghrelin has been identified as an indicator of energy insufficiency and anabolic modulator of energy homeostasis. Human studies have found a preprandial rise and postprandial decline in plasma ghrelin levels, consistent with a role for ghrelin in hunger and meal initiation. Indeed, circulating ghrelin levels are increased by food deprivation and decreased by meals, glucose load, insulin, and somatostatin. Pharmacological increases in ghrelin trigger food intake in rats or humans and decrease energy expenditure and the relative utilization of fat as an energy substrate, leading to weight gain and adiposity with chronic central administration.

### SUMMARY OF THE INVENTION

In one aspect, the present invention provides monoclonal antibodies or antigen-binding molecules (or antigen-binding fragments) which bind to ghrelin with the same binding specificity as that of an antibody produced by a hybridoma cell line with ATCC™ deposit number PTA-120176, PTA-120174 or PTA-120175. Some of the antibodies or antigen-binding fragments bind to human ghrelin. Some of the antibodies contain grafted complementarity determining regions (CDRs) from the heavy chain and/or the light chain of an antibody produced by a hybridoma cell line with ATCC™ deposit number PTA-120176, PTA-120174 or PTA-120175. Some antibodies contain the variable regions of an antibody produced by a hybridoma cell line with ATCC™ deposit number PTA-120176, PTA-120174 or PTA-120175. Some of the antibodies of the invention are produced by a hybridoma cell line with ATCC™ deposit number PTA-120176, PTA-120174 or PTA-120175.

The antibodies or antigen-binding molecules of the invention can be of murine origin. Some other anti-ghrelin antibodies or antigen-binding molecules of the invention are chimeric. In some of these embodiments, the antibody or antigen-binding molecule contains a human heavy chain constant region and a human light chain constant region. In still some other embodiments, the antibody or antigen-binding molecule is a humanized antibody or human antibody. In some embodiments, the antibody or antigen-binding molecule of the invention is a scFv fragment, an Fv fragment, an Fd fragment, an Fab fragment or an F(ab′)₂ fragment.

In a related aspect, the invention provides isolated or recombinant polynucleotides which encode a polypeptide containing the variable region of the heavy chain and/or the variable region of the light chain of an antibody produced by a hybridoma cell line with ATCC™ deposit number PTA-120176, PTA-120174 or PTA-120175. Some of the polynucleotides encode the variable region of the heavy chain or the light chain of a human antibody or a humanized antibody. In related embodiments, the invention provides hybrid cell lines that produce a monoclonal antibody that is specifically reactive with ghrelin and has the specificity of an antibody produced by a hybridoma cell line with ATCC™ deposit number PTA-120176, PTA-120174 or PTA-120175. In some other embodiments, the invention provides pharmaceutical compositions that contain a therapeutically effective amount of an anti-ghrelin antibody described herein.

In another aspect, the invention provides methods of inhibiting or slowing weight gain in a subject. The methods entail administering to the subject a pharmaceutical composition containing the combination of (1) an antibody or antigen-binding fragment that binds to ghrelin with the same binding specificity as the antibody produced by the hybridoma cell line with ATCC™ deposit number PTA-120175 and (2) an antibody or antigen-binding fragment that binds to ghrelin with the same binding specificity as the antibody produced by the hybridoma cell line with ATCC™ deposit number PTA-120174. In some embodiments, the employed antibody combination contains (1) the antibody produced by the hybridoma cell line with ATCC™ deposit number PTA-120175 (or an antigen-binding fragment thereof) and (2) an antibody produced by the hybridoma cell line with ATCC™ deposit number PTA-120174 (or an antigen-binding fragment thereof). In some of these embodiments, the administered pharmaceutical composition additionally contains a third antibody (or antigen-binding fragment) that binds to ghrelin with the same binding specificity as the antibody produced by the hybridoma cell line with ATCC™ deposit number PTA-120176. In some embodiments, the third antibody is the antibody produced by the hybridoma cell line with ATCC™ deposit number PTA-120176 (or an antigen-binding fragment thereof). Some embodiments of the invention are directed to inhibiting or slowing weight gain in human subjects. In various embodiments, the antibodies contained in the pharmaceutical composition can be murine antibodies, chimeric antibodies, humanized antibodies, or human antibodies.

In another aspect, the invention provides methods for treating obesity in a subject. The methods involve administering to the subject a pharmaceutical composition that contains (1) an antibody or antigen-binding fragment that binds to ghrelin with the same binding specificity as the antibody produced by the hybridoma cell line with ATCC™ deposit number PTA-120175 and (2) an antibody or antigen-binding fragment that binds to ghrelin with the same binding specificity as the antibody produced by the hybridoma cell line with ATCC™ deposit number PTA-120174. Some preferred embodiments of the invention are directed to treating obesity in human subjects. In some embodiments, the administered pharmaceutical composition contains (1) the antibody produced by the hybridoma cell line with ATCC™ deposit number PTA-120175 (or an antigen-binding fragment thereof), and (2) an antibody produced by the hybridoma cell line with ATCC™ deposit number PTA-120174 (or an antigen-binding fragment thereof). In some methods of the invention, the pharmaceutical composition can optionally contain a third antibody or antigen-binding fragment that binds to ghrelin with the same binding specificity as the antibody produced by the hybridoma cell line with ATCC™ deposit number PTA-120176. In some of these embodiments, the third antibody is the antibody produced by the hybridoma cell line with ATCC™ deposit number PTA-120176 (or an antigen-binding fragment thereof). In various embodiments, the administered antibodies can be murine antibodies, chimeric antibodies, humanized antibodies, or human antibodies.

### DESCRIPTION OF THE DRAWINGS

FIG. 1 shows the rate of energy expenditure (heat, top left), respiratory exchange ratio (RER, top right), the rates of carbon dioxide production (VCO₂, bottom left) and oxygen consumption (VO₂, bottom right) in food deprived, antibody-treated, adult male C57BL/6J mice as recorded in open-circuit indirect calorimetry chambers. Data are expressed in 2 h bins as M±SEM during the first 16 h of fasting beginning at light onset. Mice received subcutaneous administration (15 mg/kg total dose) of ghrelin mAbs in doublets (black ) JG2:JG4, (green □) JG3:JG4, and (blue ▴) JG2:JG3 (n=6) or (red ▾) a nicotine control Ab (n=6, NIC-1 9D9) 4 days prior to data collection; *, p<0.05 vs control Ab-treated mice. Dark onset begins at hours 13-14 of the fasting stage.

FIG. 2 shows the rate of energy expenditure (heat, top left), respiratory exchange ratio (RER, top right), the rates of carbon dioxide production (VCO₂, bottom left) and oxygen consumption (VO₂, bottom right) in antibody-treated, adult male C57BL/6J mice refeeding after a 24 h fast as recorded in open-circuit indirect calorimetry chambers. Data are expressed in 1 h bins as M±SEM during the first 6 h of refeeding beginning at light onset. Mice received subcutaneous administration (15 mg/kg total dose) of ghrelin mAbs in doublets (black ) JG2:JG4, (green ) JG3:JG4, and (blue ▴) JG2:JG3 (n=6) or (red ▾) a nicotine control Ab (n=6, NIC-1 9D9) 5 days prior to data collection; *, p<0.05, **, p<0.01 vs control Ab-treated mice.

FIG. 3 shows food intake in 24 h food-deprived, antibody-treated adult male C57BL/6J mice as recorded in open-circuit indirect calorimetry chambers. Data are expressed in 30 min bins as M±SEM during the first 6 h of refeeding beginning at light onset. Mice received subcutaneous administration (15 mg/kg total dose) of ghrelin mAbs in doublets (black ) JG2:JG4, (green ) JG3:JG4, and (blue ▴) JG2:JG3 (n=6) or (red ▾) a nicotine control Ab (n=6, NIC-1 9D9) 5 days prior to data collection.

FIG. 4 shows the rate of energy expenditure (heat, top left), respiratory exchange ratio (RER, top right), the rates of carbon dioxide production (VCO₂, bottom left) and oxygen consumption (VO2, bottom right) in antibody-treated, adult male C57BL/6J mice as recorded in open-circuit indirect calorimetry chambers. Data are expressed in 1 h bins as M±SEM during the last hour of the fasting stage (“Unfed”) and the first 6 h of refeeding beginning at light onset. Mice received subcutaneous administration (15 mg/kg total dose) of ghrelin mAbs in triplet () Ghr mAbs JG2, JG3, and JG4 (n=6) or () a nicotine control Ab (n=6, NIC-1 9D9) 5 days prior to data collection; *, p<0.05 vs control Ab-treated mice.

FIG. 5 shows food intake in 24 h food-deprived, antibody-treated adult male C57BL/6J mice as recorded in open-circuit indirect calorimetry chambers. Data are expressed in 30 min bins as M±SEM during the first 6 h of refeeding beginning at light onset. Mice received subcutaneous administration (15 mg/kg total dose) of ghrelin mAbs in triplet () Ghr mAbs JG2, JG3, and JG4 (n=6) or (□) a nicotine control Ab (n=6, NIC-1 9D9) 5 days prior to data collection; ***, p<0.0001 vs control Ab-treated mice.

### DETAILED DESCRIPTION OF THE INVENTION

### I. Overview

The present invention is predicated in part on the development by the present inventor of monoclonal antibodies that targeted acyl-ghrelin. As detailed in the Examples below, the antibodies were generated against several distinct haptens and all bound with high specificity to the active octanoylated form of ghrelin in vitro. Among the exemplified antibodies, mAb JG4 1C4 (ATCC Deposit PTA-120175) was procured against a hapten containing only the first ten residues of acyl-ghrelin along with a C-terminal cysteine used for conjugation to the carrier protein KLH. This antibody has the highest affinity to the peptide hapten (77.6 pM). It binds with high affinity to the N-terminus of full-length acyl-ghrelin as well as acyl-ghrelin fragments 1-5 and 1-10. The second antibody, JG2 mAb (ATCC Deposit PTA-120176), was prepared against a hapten containing the C-terminal ghrelin residues 13-28 and binds both acyl and des-acyl forms of ghrelin in vitro, presumably at their C-terminus. Finally, mAb JG3 (ATCC Deposit PTA-120174) was created against the full-length ghrelin peptide and, like JG2, complexes with both des-acyl-ghrelin and acyl-ghrelin.

The inventor also observed that a combination of the anti-ghrelin monoclonal antibodies is more effective in blunting fasting-induced reductions in energy expenditure and increases in food intake. Specifically, it was found that animals that were administered a combination of JG3:JG4 (termed a doublet) or JG2:JG3:JG4 (termed a triplet) demonstrated higher heat dispersion and rate of respiration (higher CO2 emission and O2 consumption) during a 24 h fast refeed. Mice administered the triplet cocktail of JG2:JG3:JG4 also demonstrated decreased food intake upon refeeding as compared to control animals. The inventor's studies indicate that an oligoclonal response provides a better means to maintain whole body energy expenditure during fasting and subsequent refeeding over a 24 h period, as well as to reduce overall food intake upon refeeding.

In accordance with results obtained from these studies, the invention provides anti-ghrelin antibodies and related pharmaceutical compositions. The antibodies and pharmaceutical compositions containing the antibodies are useful as therapeutic or prophylactic agents in treating obesity and preventing undesired weight gain. The following sections provide guidance for making and using the compositions of the invention.

### II. Definitions

Unless defined otherwise, all technical and scientific terms used herein have the same meaning as commonly understood by those of ordinary skill in the art to which this invention pertains. The following references provide one of skill with a general definition of many of the terms used in this invention: Oxford Dictionary of Biochemistry and Molecular Biology, Smith et al. (eds.), Oxford University Press (revised ed., 2000); Dictionary of Microbiology and Molecular Biology, Singleton et al. (Eds.), John Wiley & Sons (3PrdP ed., 2002); and A Dictionary of Biology (Oxford Paperback Reference), Martin and Hine (Eds.), Oxford University Press (4PthP ed., 2000). In addition, the following definitions are provided to assist the reader in the practice of the invention.

The term “antibody” and “antigen-binding molecule” is used to denote polypeptide chain(s) which exhibit a strong monovalent, bivalent or polyvalent binding to a given epitope or epitopes. Unless otherwise noted, antibodies or antigen-binding molecules of the invention can have sequences derived from any vertebrate, camelid, avian or pisces species. They can be generated using any suitable technology, e.g., hybridoma technology, ribosome display, phage display, gene shuffling libraries, semi-synthetic or fully synthetic libraries or combinations thereof. As detailed herein, antibodies or antigen-binding molecules of the invention include intact antibodies, antigen-binding polypeptide chains and other designer antibodies (see, e.g., Serafini, J Nucl Med. 34:533-6, 1993).

An intact “antibody” typically comprises at least two heavy (H) chains (about 50-70 kD) and two light (L) chains (about 25 kD) inter-connected by disulfide bonds. The recognized immunoglobulin genes encoding antibody chains include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

Each heavy chain of an antibody is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The VH and VL regions of an antibody can be further subdivided into regions of hypervariability, also termed complementarity determining regions (CDRs), which are interspersed with the more conserved framework regions (FRs). Each VH and VL is composed of three CDRs and four FRs, arranged from amino-terminus to carboxyl-terminus in the following order: FR1, CDR1, FR2, CDR2, FR3, CDR3, FR4. The locations of CDR and FR regions and a numbering system have been defined by, e.g., Kabat et al., Sequences of Proteins of Immunological Interest, U.S. Department of Health and Human Services, U.S. Government Printing Office (1987 and 1991).

Amino acids from the variable regions of the mature heavy and light chains of immunoglobulins are designated Hx and Lx respectively, where x is a number designating the position of an amino acid according to the scheme of Kabat et al, supra. Kabat et al. list many amino acid sequences for antibodies for each subgroup, and lists the most commonly occurring amino acid for each residue position in that subgroup to generate a consensus sequence. Kabat et al. use a method for assigning a residue number to each amino acid in a listed sequence, and this method for assigning residue numbers has become standard in the field. Kabat's scheme is extendible to other antibodies not included in his compendium by aligning the antibody in question with one of the consensus sequences in Kabat et al. by reference to conserved amino acids. The use of the Kabat numbering system readily identifies amino acids at equivalent positions in different antibodies. For example, an amino acid at the L50 position of a human antibody occupies the equivalent position to an amino acid position L50 of a mouse antibody. Likewise, nucleic acids encoding antibody chains are aligned when the amino acid sequences encoded by the respective nucleic acids are aligned according to the Kabat numbering convention.

Antibody or antigen-binding molecule also includes antibody fragments which contain the antigen-binding portions of an intact antibody that retain capacity to bind the cognate antigen. Examples of such antibody fragments include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); See, e.g., Bird et al., Science 242:423-426, 1988; and Huston et al., Proc. Natl. Acad. Sci. USA 85:5879-5883, 1988.

Antibodies or antigen-binding molecules of the invention further includes one or more immunoglobulin chains that are chemically conjugated to, or expressed as, fusion proteins with other proteins. It also includes bispecific antibody. A bispecific or bifunctional antibody is an artificial hybrid antibody having two different heavy/light chain pairs and two different binding sites. Other antigen-binding fragments or antibody portions of the invention include bivalent scFv (diabody), bispecific scFv antibodies where the antibody molecule recognizes two different epitopes, single binding domains (dAbs), and minibodies.

The various antibodies or antigen-binding fragments described herein can be produced by enzymatic or chemical modification of the intact antibodies, or synthesized de novo using recombinant DNA methodologies (e.g., single chain Fv), or identified using phage display libraries (see, e.g., McCafferty et al., Nature 348:552-554, 1990). For example, minibodies can be generated using methods described in the art, e.g., Vaughan and Sollazzo, Comb Chem High Throughput Screen. 4:417-30 2001. Bispecific antibodies can be produced by a variety of methods including fusion of hybridomas or linking of Fab′ fragments. See, e.g., Songsivilai & Lachmann, Clin. Exp. Immunol. 79:315-321 (1990); Kostelny et al., J. Immunol. 148:1547-1553 (1992). Single chain antibodies can be identified using phage display libraries or ribosome display libraries, gene shuffled libraries. Such libraries can be constructed from synthetic, semi-synthetic or nave and immunocompetent sources.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, as shown in the Examples below, a mouse anti-ghrelin antibody can be modified by replacing its constant region with the constant region from a human immunoglobulin. Due to the replacement with a human constant region, the chimeric antibody can retain its specificity in recognizing human ghrelin while having reduced antigenicity in human as compared to the original mouse antibody.

A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994.

Binding specificity of an antibody or antigen-binding molecule refers to the ability of the combining site of an individual antibody or antigen-binding molecule to react with only one antigenic determinant. The combining site of a typical antibody is located in the Fab portion of the molecule and is constructed from the hypervariable regions of the heavy and light chains. Binding affinity is the strength of the reaction between a single antigenic determinant and a single combining site on the antibody or antigen-binding molecule. It is the sum of the attractive and repulsive forces operating between the antigenic determinant and the combining site. Affinity is the equilibrium constant that describes the antigen-antibody reaction.

The phrase “specifically (or selectively) bind to” refers to a binding reaction between an antibody or antigen-binding molecule (e.g., an anti-ghrelin antibody) and a cognate antigen (e.g., a human ghrelin polypeptide) in a heterogeneous population of proteins and other biologics. The phrases “an antibody recognizing an antigen” and “an antibody specific for an antigen” are used interchangeably herein with the term “an antibody which binds specifically to an antigen”.

The term “epitope” means a protein determinant capable of specific binding to an antibody. Epitopes usually consist of chemically active surface groupings of molecules such as amino acids or sugar side chains and usually have specific three dimensional structural characteristics, as well as specific charge characteristics. Conformational and nonconformational epitopes are distinguished in that the binding to the former but not the latter is lost in the presence of denaturing solvents.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “conservatively modified variant” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. The following eight groups contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

8) Cysteine (C), Methionine (M) (see, e.g., Creighton, Proteins (1984)).

The terms “identical” or percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or more preferably over a region that is 100 to 500 or 1000 or more nucleotides (or 20, 50, 200 or more amino acids) in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

The term “operably linked” refers to a functional relationship between two or more polynucleotide (e.g., DNA) segments. Typically, it refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adenoassociated viruses), which serve equivalent functions.

The term “recombinant host cell” (or simply “host cell”) refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

The term “treating” includes the administration of compounds or agents to prevent or delay the onset of the symptoms, complications, or biochemical indicia of a disease (e.g., a tumor), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease.

### III. Anti-Ghrelin Antibodies Derived from Exemplified Antibodies JG2, JG3 and JG4

The invention provides monoclonal antibodies or antigen-binding fragments or molecules that specifically bind to and sequester ghrelin protein or peptide thereof. These anti-ghrelin agents are capable of suppressing ghrelin mediated signaling or cellular activities, e.g., ghrelin induced food intake as described in the Examples below. The anti-ghrelin antibodies of the invention are derived from the exemplified antibodies, JG2 (produced by the hybridoma with ATCC™ deposit number PTA-120176), JG3 (produced by the hybridoma with ATCC™ deposit number PTA-120174), and JG4 (produced by the hybridoma with ATCC™ deposit number PTA-120175). Typically, anti-ghrelin antibodies of the invention have the same or substantially identical binding specificity as that of one of the exemplified antibodies. In addition, the antibodies also have similar or better binding affinity relative to the exemplified antibodies from which they are derived.

Various monoclonal antibodies or antigen-binding fragments with similar binding activities to that of the anti-ghrelin antibodies exemplified herein can be produced. General methods for preparation of monoclonal or polyclonal antibodies are well known in the art. See, e.g., Harlow & Lane, Using Antibodies, A Laboratory Manual, Cold Spring Harbor Laboratory Press, Cold Spring Harbor, N.Y., 1998; Kohler & Milstein, Nature 256:495-497, 1975; Kozbor et al., Immunology Today 4:72, 1983; and Cole et al., pp. 77-96 in Monoclonal Antibodies and Cancer Therapy, 1985. The anti-ghrelin antibodies of the invention can be generated by any technique for producing monoclonal antibody well known in the art, e.g., viral or oncogenic transformation of B lymphocytes. One animal system for preparing hybridomas is the murine system. Hybridoma production in the mouse is a very well-established procedure. After immunization an animal with an appropriate antigen (e.g., ghrelin peptide fused to a carrier protein), B cells isolated from the animal are then fused to myeloma cells to generate antibody-producing hybridomas. Monoclonal mouse anti-ghrelin antibodies can be obtained by screening the hybridomas in an ELISA assay using a ghrelin polypeptide or fusion protein. Immunization protocols and techniques for isolation of immunized splenocytes for fusion are known in the art. Fusion partners (e.g., murine myeloma cells) and fusion procedures are also well known in the art, e.g., Harlow & Lane, supra.

A typical intact antibody interacts with target antigen predominantly through amino acid residues that are located in the six heavy and light chain complimentarity determining regions (CDR's). Typically, the anti-ghrelin antibodies of the invention have at least one of their heavy chain CDR sequences or light chain CDR sequences identical to the corresponding CDR sequences of one of the exemplified antibodies (e.g., JG4). Some of these anti-ghrelin antibodies of the invention have the same binding specificity as that of the exemplified mouse anti-ghrelin antibody (clone JG4) described in the Examples below. These antibodies can compete with the mouse anti-ghrelin antibody (e.g., JG4 or JG3) for binding to ghrelin. Some anti-ghrelin antibodies of the invention have all CDR sequences in their variable regions of the heavy chain and light chain respectively identical to the corresponding CDR sequences of one of the exemplified antibodies.

In addition to having CDR sequences respectively identical to the corresponding CDR sequences of the exemplified mouse anti-ghrelin antibody (e.g., JG4 or JG3), some of the anti-ghrelin antibodies of the invention have their entire heavy chain and light chain variable region sequences respectively identical to the corresponding variable region sequences of the exemplified antibodies. In some other embodiments, other than the identical CDR sequences, the antibodies contain amino acid residues in the framework portions of the variable regions that are different from the corresponding amino acid residues of one of the exemplified antibodies (e.g., some of the humanized anti-ghrelin antibodies described below). Nevertheless, these antibodies typically have their entire variable region sequences that are substantial identical (e.g., 75%, 85%, 90%, 95%, or 99%) to the corresponding variable region sequences of the exemplified mouse anti-ghrelin antibody.

The anti-ghrelin antibodies of the invention can be an intact antibody which contains two heavy chains and two light chains. They can also be antigen-binding molecules of an intact antibody or single chain antibodies. The anti-ghrelin antibodies of the invention include antibodies produced in a non-human animal (e.g., the mouse anti-ghrelin antibody JG2, JG3 and JG4). They also include modified antibodies which are modified forms of the exemplified mouse antibodies. Often, the modified antibodies are recombinant antibodies which have similar or improved properties relative to that of one of the exemplified mouse antibodies. For example, the mouse anti-ghrelin antibody exemplified herein can be modified by deleting the constant region and replacing it with a different constant region that can lead to increased half-life, e.g., serum half-life, stability or affinity of the antibody. The modified antibodies can be created, e.g., by constructing expression vectors that include the CDR sequences from the mouse antibody grafted onto framework sequences from a different antibody with different properties (Jones et al. 1986, Nature 321, 522-525). Such framework sequences can be obtained from public DNA databases (e.g., from www.kabatdatabase.com).

### IV. Modified Anti-Ghrelin Antibodies

Some embodiments of the invention are directed to modified antibodies that are based on or modified from the mouse anti-ghrelin antibodies exemplified herein, antibodies JG2, JG3 and JG4 which respectively are produced by hybridoma cell lines with ATCC™ deposit numbers PTA-120176, PTA-120174 and PTA-120175. These include, e.g., chimeric, humanized and human anti-ghrelin antibodies. Relative to the exemplified antibody, these modified antibodies can have similar binding specificity, as well as improved binding affinity. They also have substantially reduced antigenicity when used in vivo in a non-mouse subject, e.g., a human subject. Some of the modified antibodies are chimeric antibodies which contain partial human immunoglobulin sequences (e.g., constant regions) and partial non-human immunoglobulin sequences (e.g., the variable region sequences of mouse antibody JG2, JG3 or JG4). Some other modified antibodies are humanized antibodies. Generally, a humanized antibody has one or more amino acid residues introduced into it from a source that is non-human. Methods for humanizing non-human antibodies are well known in the art, e.g., U.S. Pat. Nos. 5,585,089 and 5,693,762; Jones et al., Nature 321: 522-25, 1986; Riechmann et al., Nature 332: 323-27, 1988; and Verhoeyen et al., Science 239: 1534-36, 1988. These methods can be readily employed to generate humanized anti-ghrelin antibodies of the invention by substituting at least a portion of a CDR from a non-human anti-ghrelin antibody for the corresponding regions of a human antibody. In some embodiments, the humanized anti-ghrelin antibodies of the invention have all three CDRs in each immunoglobulin chain from the exemplified mouse anti-ghrelin antibody grafted into corresponding human framework regions.

The anti-ghrelin antibodies described above can undergo non-critical amino-acid substitutions, additions or deletions in both the variable and constant regions without loss of binding specificity or effector functions, or intolerable reduction of binding affinity. Usually, antibodies incorporating such alterations exhibit substantial sequence identity to a reference antibody (e.g., mouse anti-ghrelin antibody JG4) from which they were derived. For example, the mature light chain variable regions of some of the anti-ghrelin antibodies of the invention have at least 75% or at least 85% sequence identity to the sequence of the mature light chain variable region of the exemplified mouse anti-ghrelin antibodies. Similarly, the mature heavy chain variable regions of the antibodies typically show at least 75% or at least 85% sequence identity to the sequence of the mature heavy chain variable region of the exemplified anti-ghrelin antibodies. Some of the modified anti-ghrelin antibodies have the same specificity and increased affinity compared with the exemplified mouse anti-ghrelin antibodies. Usually, the affinity of the modified anti-ghrelin antibodies is within a factor of 2, 5, 10 or 50 of the reference mouse anti-ghrelin antibody.

Some of the anti-ghrelin antibodies of the invention are chimeric (e.g., mouse/human) antibodies which are made up of regions from a non-human anti-ghrelin antibody together with regions of human antibodies. For example, a chimeric H chain can comprise the antigen binding region of the heavy chain variable region of the mouse anti-ghrelin antibody exemplified herein (e.g., JG2, JG3 or JG4) linked to at least a portion of a human heavy chain constant region. This chimeric heavy chain may be combined with a chimeric L chain that comprises the antigen binding region of the light chain variable region of the exemplified mouse anti-ghrelin antibody linked to at least a portion of the human light chain constant region.

Chimeric anti-ghrelin antibodies of the invention can be produced in accordance with methods known in the art. For example, a gene encoding the heavy chain or light chain of a murine anti-ghrelin antibody or antigen-binding molecule can be digested with restriction enzymes to remove the murine Fc region, and substituted with the equivalent portion of a gene encoding a human Fc constant region. Expression vectors and host cells suitable for expression of recombinant antibodies and humanized antibodies in particular, are well known in the art. Vectors expressing chimeric genes encoding anti-ghrelin immunoglobulin chains can be constructed using standard recombinant techniques, e.g., Sambrook et al., Molecular Cloning: A Laboratory Manual, Cold Spring Harbor Press (3^{rd} ed., 2001); and Brent et al., Current Protocols in Molecular Biology, John Wiley & Sons, Inc. (Ringbou, ed., 2003). Human constant region sequences can be selected from various reference sources, including but not limited to those listed in Kabat et al., Sequences of Proteins of Immunological Interest, Fifth Edition, U.S. Department of Health and Human Services, U.S. Government Printing Office, 1991. More specific teachings of producing chimeric antibodies by DNA recombination have also been taught in the art, e.g., Robinson et al., International Patent Publication PCT/US86/02269; Akira et al., European Patent Application 184,187; Taniguchi, M., European Patent Application 171,496; Morrison et al., European Patent Application 173,494; Neuberger et al., International Application WO 86/01533; Cabilly et al. U.S. Pat. No. 4,816,567; Cabilly et al., European Patent Application 125,023; Better et al., Science 240:1041-1043, 1988; Liu et al., PNAS 84:3439-3443, 1987; Liu et al., J. Immunol. 139:3521-3526, 1987; Sun et al., PNAS 84:214-218, 1987; Nishimura et al., Canc. Res. 47:999-1005, 1987; Wood et al., Nature 314:446-449, 1985; Shaw et al., J. Natl. Cancer Inst. 80:1553-1559, 1988.

Chimeric antibodies which have the entire variable regions from a non-human antibody can be further humanized to reduce antigenicity of the antibody in human. This is typically accomplished by replacing certain sequences or amino acid residues in the Fv variable regions (framework regions or non-CDR regions) with equivalent sequences or amino acid residues from human Fv variable regions. These additionally substituted sequences or amino acid residues are usually not directly involved in antigen binding. More often, humanization of a non-human antibody proceeds by substituting only the CDRs of a non-human antibody (e.g., the mouse anti-ghrelin antibodies exemplified herein) for the CDRs in a human antibody. In some cases, this is followed by replacing some additional residues in the human framework regions with the corresponding residues from the non-human donor antibody. Such additional grafting is often needed to improve binding to the antigen. This is because humanized antibodies which only have CDRs grafted from a non-human antibody can have less than perfect binding activities as compared to that of the non-human donor antibody. Thus, in addition to the CDRs, humanized anti-ghrelin antibodies of the invention can often have some amino acids residues in the human framework region replaced with corresponding residues from the non-human donor antibody (e.g., the mouse antibody exemplified herein). Methods for generating humanized antibodies by CDR substitution, including criteria for selecting framework residues for replacement, are well known in the art. For example, in addition to the above noted art relating to producing chimeric antibodies, additional teachings on making humanized antibodies are provided in, e.g., Winter et al., UK Patent Application GB 2188638A (1987), U.S. Pat. No. 5,225,539; Jones et al., Nature 321:552-525, 1986; Verhoeyan et al., Science 239:1534, 1988; and Beidler et al., J. Immunol. 141:4053-4060, 1988. CDR substitution can also be carried out using oligonucleotide site-directed mutagenesis as described in, e.g., WO 94/10332 entitled “Humanized Antibodies to Fc Receptors for Immunoglobulin G on Human Mononuclear Phagocytes.”

The chimeric or humanized anti-ghrelin antibodies of the invention may be monovalent, divalent, or polyvalent immunoglobulins. For example, a monovalent chimeric antibody is a dimer (HL) formed by a chimeric H chain associated through disulfide bridges with a chimeric L chain, as noted above. A divalent chimeric antibody is a tetramer (H₂ L₂) formed by two HL dimers associated through at least one disulfide bridge. A polyvalent chimeric antibody is based on an aggregation of chains.

In addition to chimeric or humanized anti-ghrelin antibodies, also included in the invention are fully human antibodies that exhibit the same binding specificity and comparable or better binding affinity. For example, the human anti-ghrelin antibodies can have the same or better binding characteristics (e.g., binding specificity and/or binding affinity) relative to that of a reference nonhuman anti-ghrelin antibody. The reference nonhuman antibody can be any one of antibodies JG2, JG3 and JG4, which respectively are produced by hybridoma cell lines with ATCC™ deposit numbers PTA-120176, PTA-120174 and PTA-120175. Compared to the chimeric or humanized antibodies, the human anti-ghrelin antibodies of the invention have further reduced antigenicity when administered to human subjects.

The human anti-ghrelin antibodies can be generated using methods that are known in the art. For example, an in vivo method for replacing a nonhuman antibody variable region with a human variable region in an antibody while maintaining the same or providing better binding characteristics relative to that of the nonhuman antibody has been disclosed in U.S. patent application Ser. No. 10/778,726 (Publication No. 20050008625). The method replies on epitope guided replacement of variable regions of a non-human reference antibody with a fully human antibody. The resulting human antibody is generally unrelated structurally to the reference nonhuman antibody, but binds to the same epitope on the same antigen as the reference antibody. Briefly, the serial epitope-guided complementarity replacement approach is enabled by setting up a competition in cells between a “competitor” and a library of diverse hybrids of the reference antibody (“test antibodies”) for binding to limiting amounts of antigen in the presence of a reporter system which responds to the binding of test antibody to antigen. The competitor can be the reference antibody or derivative thereof such as a single-chain Fv fragment. The competitor can also be a natural or artificial ligand of the antigen which binds to the same epitope as the reference antibody. The only requirements of the competitor are that it binds to the same epitope as the reference antibody, and that it competes with the reference antibody for antigen binding. The test antibodies have one antigen-binding V-region in common from the nonhuman reference antibody, and the other V-region selected at random from a diverse source such as a repertoire library of human antibodies. The common V-region from the reference antibody serves as a guide, positioning the test antibodies on the same epitope on the antigen, and in the same orientation, so that selection is biased toward the highest antigen-binding fidelity to the reference antibody.

Many types of reporter system can be used to detect desired interactions between test antibodies and antigen. For example, complementing reporter fragments may be linked to antigen and test antibody, respectively, so that reporter activation by fragment complementation only occurs when the test antibody binds to the antigen. When the test antibody- and antigen-reporter fragment fusions are co-expressed with a competitor, reporter activation becomes dependent on the ability of the test antibody to compete with the competitor, which is proportional to the affinity of the test antibody for the antigen. Other reporter systems that can be used include the reactivator of an auto-inhibited reporter reactivation system (RAIR) as disclosed in U.S. patent application Ser. No. 10/208,730 (Publication No. 20030198971), or competitive activation system disclosed in U.S. patent application Ser. No. 10/076,845 (Publication No. 20030157579).

With the serial epitope-guided complementarity replacement system, selection is made to identify cells expresses a single test antibody along with the competitor, antigen, and reporter components. In these cells, each test antibody competes one-on-one with the competitor for binding to a limiting amount of antigen. Activity of the reporter is proportional to the amount of antigen bound to the test antibody, which in turn is proportional to the affinity of the test antibody for the antigen and the stability of the test antibody. Test antibodies are initially selected on the basis of their activity relative to that of the reference antibody when expressed as the test antibody. The result of the first round of selection is a set of “hybrid” antibodies, each of which is comprised of the same non-human V-region from the reference antibody and a human V-region from the library, and each of which binds to the same epitope on the antigen as the reference antibody. One of more of the hybrid antibodies selected in the first round will have an affinity for the antigen comparable to or higher than that of the reference antibody.

In the second V-region replacement step, the human V-regions selected in the first step are used as guide for the selection of human replacements for the remaining non-human reference antibody V-region with a diverse library of cognate human V-regions. The hybrid antibodies selected in the first round may also be used as competitors for the second round of selection. The result of the second round of selection is a set of fully human antibodies which differ structurally from the reference antibody, but which compete with the reference antibody for binding to the same antigen. Some of the selected human antibodies bind to the same epitope on the same antigen as the reference antibody. Among these selected human antibodies, one or more binds to the same epitope with an affinity which is comparable to or higher than that of the reference antibody.

Using one of the mouse or chimeric anti-ghrelin antibodies described above as the reference antibody, this method can be readily employed to generate human antibodies that bind to human ghrelin with the same binding specificity and the same or better binding affinity. In addition, such human anti-ghrelin antibodies can also be commercially obtained from companies which customarily produce human antibodies, e.g., KaloBios, Inc. (Mountain View, Calif.). To obtain human antibodies with the same or better affinities for a specific epitope than a starting non-human antibody (e.g., a mouse anti-ghrelin antibody), the KaloBios, Inc. technologies employ a human “acceptor” antibody library. A directed or epitope focused library of human antibodies which bind to the identical epitope as the non-human antibody, though with varying affinities, is then generated. Antibodies in the epitope focused library are then selected for similar or higher affinity than that of the starting non-human antibody. The identified human antibodies are then subject to further analysis for affinity and sequence identity.

The anti-ghrelin antibodies or antigen-binding molecules of the invention also include single chain antibodies, bispecific antibodies and multi-specific antibodies. In some embodiments, the antibodies of the invention are single chain antibodies. Single chain antibodies contain in a single stably-folded polypeptide chain the antigen-binding regions from both the heavy chain and the light chain. As such, single chain antibodies typically retain the binding specificity and affinity of monoclonal antibodies but are of considerably small size than classical immunoglobulins. For certain applications, the anti-ghrelin single chain antibodies of the invention may provide many advantageous properties as compared to an intact anti-ghrelin antibody. These include, e.g., faster clearance from the body, greater tissue penetration for both diagnostic imaging and therapy, and a significant decrease in immunogenicity when compared with mouse-based antibodies. Other potential benefits of using single chain antibodies include enhanced screening capabilities in high throughput screening methods and the potential for non-parenteral application. Single chain anti-ghrelin antibodies of the invention can be prepared using methods that have been described in the art. Examples of such techniques include those described in U.S. Pat. Nos. 4,946,778 and 5,258,498; Huston et al., Methods in Enzymology 203:46-88, 1991; Shu et al., Proc. Natl. Acad. Sci. USA 90:7995-7999, 1993; and Skerra et al., Science 240:1038-1040, 1988.

### V. Polynucleotides, Vectors and Host Cells for Producing Anti-Ghrelin Antibodies

The invention provides substantially purified polynucleotides (DNA or RNA) which encode polypeptides comprising segments or domains of the anti-ghrelin antibody chains or antigen-binding molecules described above. Some of the polynucleotides of the invention comprise the nucleotide sequence encoding the heavy chain variable region of exemplified mouse anti-ghrelin antibody JG2, JG3 or JG4. They can alternatively or additionally comprise the nucleotide sequence encoding the light chain variable region of the described anti-ghrelin antibody. Some other polynucleotides of the invention comprise nucleotide sequences that are substantially identical (e.g., at least 65, 80%, 95%, or 99%) to the nucleotide sequence encoding the heavy chain variable region or light chain variable region of an exemplified anti-ghrelin antibody (e.g., antibody JG4). Also provided in the invention are polynucleotides which encode at least one CDR region and usually all three CDR regions from the heavy or light chain of a described anti-ghrelin antibody, e.g., mouse anti-ghrelin antibody JG4 or JG3. Because of the degeneracy of the code, a variety of nucleic acid sequences will encode each of the immunoglobulin amino acid sequences. When expressed from appropriate expression vectors, polypeptides encoded by these polynucleotides are capable of exhibiting antigen binding capacity.

The polynucleotides of the invention can encode only the variable region sequence of an anti-ghrelin antibody. They can also encode both a variable region and a constant region of the antibody. Some of polynucleotide sequences of the invention encode a mature heavy chain variable region sequence that is substantially identical (e.g., at least 80%, 90%, or 99%) to the mature heavy chain variable region sequence of an anti-ghrelin antibody described herein (e.g., mouse antibody JG4). Some other polynucleotide sequences encode a mature light chain variable region sequence that is substantially identical to the mature light chain variable region sequence of the described anti-ghrelin antibody. Some of the polynucleotide sequences encode a polypeptide that comprises variable regions of both the heavy chain and the light chain of an exemplified anti-ghrelin antibody, e.g., mouse anti-ghrelin antibody JG4. Some other polynucleotides encode two polypeptide segments that respectively are substantially identical to the variable regions of the heavy chain and the light chain of one of the disclosed anti-ghrelin antibodies.

The polynucleotide sequences can be produced by de novo solid-phase DNA synthesis or by PCR mutagenesis of an existing sequence (e.g., sequences as described in the Examples below) encoding an anti-ghrelin antibody or its binding fragment. Direct chemical synthesis of nucleic acids can be accomplished by methods known in the art, such as the phosphotriester method of Narang et al., Meth. Enzymol. 68:90, 1979; the phosphodiester method of Brown et al., Meth. Enzymol. 68:109, 1979; the diethylphosphoramidite method of Beaucage et al., Tetra. Lett., 22:1859, 1981; and the solid support method of U.S. Pat. No. 4,458,066. Introducing mutations to a polynucleotide sequence by PCR can be performed as described in, e.g., PCR Technology: Principles and Applications for DNA Amplification, H.A. Erlich (Ed.), Freeman Press, NY, N.Y., 1992; PCR Protocols: A Guide to Methods and Applications, Innis et al. (Ed.), Academic Press, San Diego, Calif., 1990; Mattila et al., Nucleic Acids Res. 19:967, 1991; and Eckert et al., PCR Methods and Applications 1:17, 1991.

Also provided in the invention are expression vectors and host cells for producing the anti-ghrelin antibodies described above. Various expression vectors can be employed to express the polynucleotides encoding the anti-ghrelin antibody chains or binding fragments. Both viral-based and nonviral expression vectors can be used to produce the antibodies in a mammalian host cell. Nonviral vectors and systems include plasmids, episomal vectors, typically with an expression cassette for expressing a protein or RNA, and human artificial chromosomes (see, e.g., Harrington et al., Nat. Genet. 15:345, 1997). For example, nonviral vectors useful for expression of the anti-ghrelin polynucleotides and polypeptides in mammalian (e.g., human) cells include pThioHis A, B & C, pcDNA3.1/His, pEBVHis A, B & C (Invitrogen, San Diego, Calif.), MPSV vectors, and numerous other vectors known in the art for expressing other proteins. Useful viral vectors include vectors based on retroviruses, adenoviruses, adenoassociated viruses, herpes viruses, vectors based on SV40, papilloma virus, HBP Epstein Barr virus, vaccinia virus vectors and Semliki Forest virus (SFV). See, Brent et al., supra; Smith, Annu. Rev. Microbiol. 49:807, 1995; and Rosenfeld et al., Cell 68:143, 1992.

The choice of expression vector depends on the intended host cells in which the vector is to be expressed. Typically, the expression vectors contain a promoter and other regulatory sequences (e.g., enhancers) that are operably linked to the polynucleotides encoding an anti-ghrelin antibody chain or fragment. In some embodiments, an inducible promoter is employed to prevent expression of inserted sequences except under inducing conditions. Inducible promoters include, e.g., arabinose, lacZ, metallothionein promoter or a heat shock promoter. Cultures of transformed organisms can be expanded under noninducing conditions without biasing the population for coding sequences whose expression products are better tolerated by the host cells. In addition to promoters, other regulatory elements may also be required or desired for efficient expression of an anti-ghrelin antibody chain or fragment. These elements typically include an ATG initiation codon and adjacent ribosome binding site or other sequences. In addition, the efficiency of expression may be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer may be used to increase expression in mammalian host cells.

The expression vectors may also provide a secretion signal sequence position to form a fusion protein with polypeptides encoded by inserted anti-ghrelin antibody sequences. More often, the inserted anti-ghrelin antibody sequences are linked to a signal sequences before inclusion in the vector. Vectors to be used to receive sequences encoding anti-ghrelin antibody light and heavy chain variable domains sometimes also encode constant regions or parts thereof. Such vectors allow expression of the variable regions as fusion proteins with the constant regions thereby leading to production of intact antibodies or fragments thereof. Typically, such constant regions are human.

The host cells for harboring and expressing the anti-ghrelin antibody chains can be either prokaryotic or eukaryotic. E. coli is one prokaryotic host useful for cloning and expressing the polynucleotides of the present invention. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express anti-ghrelin polypeptides of the invention. Insect cells in combination with baculovirus vectors can also be used.

In some preferred embodiments, mammalian host cells are used to express and produce the anti-ghrelin polypeptides of the present invention. For example, they can be either a hybridoma cell line expressing endogenous immunoglobulin genes (e.g., the myeloma hybridoma clones as described in the Examples) or a mammalian cell line harboring an exogenous expression vector (e.g., the SP2/0 myeloma cells exemplified below). These include any normal mortal or normal or abnormal immortal animal or human cell. For example, a number of suitable host cell lines capable of secreting intact immunoglobulins have been developed, including the CHO cell lines, various Cos cell lines, HeLa cells, myeloma cell lines, transformed B-cells and hybridomas. The use of mammalian tissue cell culture to express polypeptides is discussed generally in, e.g., Winnacker, From Genes to Clones, VCH Publishers, N.Y., N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

Methods for introducing expression vectors containing the polynucleotide sequences of interest vary depending on the type of cellular host. For example, calcium chloride transfection is commonly utilized for prokaryotic cells, whereas calcium phosphate treatment or electroporation may be used for other cellular hosts (see generally Sambrook et al., supra). Other methods include, e.g., electroporation, calcium phosphate treatment, liposome-mediated transformation, injection and microinjection, ballistic methods, virosomes, immunoliposomes, polycation:nucleic acid conjugates, naked DNA, artificial virions, fusion to the herpes virus structural protein VP22 (Elliot and O'Hare, Cell 88:223, 1997), agent-enhanced uptake of DNA, and ex vivo transduction. For long-term, high-yield production of recombinant proteins, stable expression will often be desired. For example, cell lines which stably express anti-ghrelin antibody chains or binding fragments can be prepared using expression vectors of the invention which contain viral origins of replication or endogenous expression elements and a selectable marker gene. Following introduction of the vector, cells may be allowed to grow for 1-2 days in an enriched media before they are switched to selective media. The purpose of the selectable marker is to confer resistance to selection, and its presence allows growth of cells which successfully express the introduced sequences in selective media. Resistant, stably transfected cells can be proliferated using tissue culture techniques appropriate to the cell type.

### VI. Therapeutic Applications and Pharmaceutical Compositions

The anti-ghrelin antibodies described herein can be employed in many therapeutic or prophylactic applications by antagonizing or sequestering the ghrelin protein in subjects suffering from or at the risk of developing a condition or disorder mediated by or associated with ghrelin (e.g., obesity). These include, but are not limited to, decreasing adiposity and treating obesity, preventing the development of obesity, reversing or slowing of weight gain, decreasing feed efficiency, inhibiting restriction induced feeding during low-calorie diet-induced weight loss, and sparing lean body mass in a subject during low-calorie diet-induced weight loss.

Preferably, therapeutic applications of the invention employ a combination of antibodies (or antigen-binding fragments or molecules) that are derived from the antibodies exemplified herein (JG4, JG3, and JG2). The administered pharmaceutical composition can contain amounts of the antibodies in various ratios as appropriate. For example, the composition can contain one antibody in a ratio of 1:1, 1.25:1, 1.5:1, 1.75:1, 2:1, 3:1, 4:1, 5:1 or even higher relative to one or more other antibodies in the composition. In some preferred embodiments, the antibodies are present in the pharmaceutical compositions in equal ratios, e.g., 1:1 for doublets or 1:1:1 for triplets. As demonstrated in the Examples herein, a “doublet” treatment that combined JG4 with a particular C-terminally directed anti-ghrelin antibody (JG3) maintained greater energy expenditure and utilization of carbohydrate as a fuel substrate in fasted mice. Moreover, a “triplet” mAb cocktail combination that included the additional C-terminally directed antibody (JG2) that had been comparably ineffective in conjunction with JG3 or JG4 alone not only promoted increased energy expenditure but also reduced deprivation-induced food intake. The JG3:JG4 complex may have altered secondary structure of the ghrelin peptide, which could significantly reduce positive interaction at the GHSR1a, due to JG3's ability to bind ghrelin only in a conformational manner as demonstrated by BIAcore analysis. The mAb doublet JG3:JG4 bound ghrelin in a manner that increased relative energy expenditure and the relative utilization of carbohydrate as a fuel source in fasted C57BL/6J mice.

Some therapeutic applications of the invention employ the combination of an antibody derived from antibody JG4 (produced by ATCC Deposit No. PTA-120175) and an antibody derived from antibody JG3 (produced by ATCC Deposit No. PTA-120174). The employed antibodies typically have the same or substantially identical binding specificity as that of the exemplified antibodies from which they are derived. In addition, the employed antibodies also have similar or better binding affinity relative to that of the exemplified antibodies. In some embodiments, the employed antibody combination contains antibody JG4 produced by ATCC Deposit No. PTA-120175 (or an antigen-binding fragment thereof) and antibody JG3 produced by ATCC Deposit No. PTA-120174 (or an antigen-binding fragment thereof). In some embodiments, other than the JG3- and JG4-derived antibody doublet, the employed antibody combination contains a third antibody that is derived from antibody JG2 (produced by ATCC Deposit No. PTA-120176). In some preferred embodiments, therapeutic applications of the invention employ a triplet of the JG4, JG3 and JG2 antibodies. Antibodies employed in the methods of the invention can be a full length antibody or an antigen-binding fragment or molecule described above. The antibodies can be mouse antibodies, chimeric antibodies, humanized antibodies or fully human antibodies.

Some embodiments of the invention employ a pharmaceutical composition containing the above-described anti-ghrelin antibody combination for administration to a subject already affected by a disease or condition caused by or associated with ghrelin (e.g., obesity). The composition contains the antibody or antigen-binding molecules in an amount sufficient to cure, partially arrest, or detectably slow the progression of the condition, and its complications. In prophylactic applications, compositions containing the anti-ghrelin antibodies or antigen-binding molecules are administered to a subject not already suffering from a ghrelin-related disorder. Rather, they are directed to a subject who is at the risk of, or has a predisposition, to developing such a disorder. Such applications allow the subject to enhance the subject's resistance or to retard the progression of a disorder mediated by ghrelin.

The invention provides pharmaceutical compositions comprising the anti-ghrelin antibodies or antigen-binding molecules formulated together with a pharmaceutically acceptable carrier. The compositions can additionally contain other therapeutic agents that are suitable for treating or preventing a given disorder. Pharmaceutically carriers enhance or stabilize the composition, or to facilitate preparation of the composition. Pharmaceutically acceptable carriers include solvents, dispersion media, coatings, antibacterial and antifungal agents, isotonic and absorption delaying agents, and the like that are physiologically compatible.

A pharmaceutical composition of the present invention can be administered by a variety of methods known in the art. The route and/or mode of administration vary depending upon the desired results. It is preferred that administration be intravenous, intramuscular, intraperitoneal, or subcutaneous, or administered proximal to the site of the target. The pharmaceutically acceptable carrier should be suitable for intravenous, intramuscular, subcutaneous, parenteral, spinal or epidermal administration (e.g., by injection or infusion). Depending on the route of administration, the active compound, i.e., antibody, bispecific and multispecific molecule, may be coated in a material to protect the compound from the action of acids and other natural conditions that may inactivate the compound.

The composition should be sterile and fluid. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. Long-term absorption of the injectable compositions can be brought about by including in the composition an agent which delays absorption, for example, aluminum monostearate or gelatin.

Pharmaceutical compositions of the invention can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Typically, a therapeutically effective dose or efficacious dose of the anti-ghrelin antibody is employed in the pharmaceutical compositions of the invention. The anti-ghrelin antibodies are formulated into pharmaceutically acceptable dosage forms by conventional methods known to those of skill in the art. Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

Actual dosage levels of the active ingredients (e.g., total amount of the anti-ghrelin antibodies) in the pharmaceutical compositions of the present invention can be varied so as to obtain an amount of the active ingredient which is effective to achieve the desired therapeutic response for a particular patient, composition, and mode of administration, without being toxic to the patient. The selected dosage level depends upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors.

A physician or veterinarian can start doses of each of the antibodies employed in the pharmaceutical composition at levels lower than that required to achieve the desired therapeutic effect and gradually increase the dosage until the desired effect is achieved. In general, effective doses of the compositions of the present invention vary depending upon many different factors, including the specific disease or condition to be treated, means of administration, target site, physiological state of the patient, whether the patient is human or an animal, other medications administered, and whether treatment is prophylactic or therapeutic. Treatment dosages need to be titrated to optimize safety and efficacy. For administration with an antibody, the dosage ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once per every two weeks or once a month or once every 3 to 6 months.

The antibody combination of the invention is usually administered on multiple occasions. Intervals between single dosages can be weekly, monthly or yearly. Intervals can also be irregular as indicated by measuring blood levels of anti-ghrelin antibody in the patient. In some methods, dosage is adjusted to achieve a plasma antibody concentration of 1-1000 μg/ml and in some methods 25-300 μg/ml. Alternatively, antibody can be administered as a sustained release formulation, in which case less frequent administration is required. Dosage and frequency vary depending on the half-life of the antibody in the patient. In general, humanized antibodies show longer half life than that of chimeric antibodies and nonhuman antibodies. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

### VII. Deposit of Materials

Murine hybridoma 7B4, 8H11, and 1C4 were deposited with and tested by the American Type Culture Collection, Manassas, Va., USA (ATCC®) on May 3, 2013, and have been assigned the ATCC® Patent Deposit Designation PTA-120176, PTA-120174 and PTA-120175, respectively. The deposits provide cell lines that express mouse anti-ghrelin antibody JG2, JG3 and JG4, respectively. The deposits were made under the provisions of the Budapest Treaty on the International Recognition of the Deposit of Microorganisms for the Purpose of Patent Procedure and the Regulations thereunder (Budapest Treaty). This assures maintenance of a viable cell culture for 30 years from the date of the deposit for each of the deposits. The cell lines will be made available by ATCC under the terms of the Budapest Treaty which assures permanent and unrestricted availability of the progeny of the culture to the public upon issuance of the pertinent U.S. patent, and assures availability of the progeny to one determined by the U.S. Commissioner of Patents and Trademarks to be entitled thereto according to 35 U.S.C. §122 and the Commissioner's rules pursuant thereto (including 37 CFR §1.14 with particular reference to 886 OG 638). The assignee of the present application agreed that, subject to 37 CFR 1.808(b), all restrictions imposed by the depositor on the availability to the public of the deposited biological material be irrevocably removed upon the granting of the patent. The assignee of the present application has also agreed that if the cell culture deposits should die or be lost or destroyed when cultivated under suitable conditions, it will be promptly replaced on notification with a viable specimen of the same culture. Availability of the deposit is not to be construed as a license to practice the invention in contravention of the rights granted under the authority of any government in accordance with its patent laws.

### EXAMPLES

The following examples are provided to further illustrate the invention but not to limit its scope. Other variants of the invention will be readily apparent to one of ordinary skill in the art and are encompassed by the appended claims.

### Example 1

### Monoclonal Antibodies Bind with High Specificity to Acyl-Ghrelin

Monoclonal antibody affinity to full-length acyl-ghrelin was initially tested by ELISA. JG4 1C4 had the highest affinity to the peptide with an estimated Kd of 6.2 nM. JG3 8H11 and JG2 7B4 mAbs had relative affinities estimated by ELISA of 50 nM and 800 nM respectively. Noteworthy was that mAb JG4 failed to bind des-acyl-ghrelin up to 3 μM, whereas the other two mAbs demonstrated binding as potent as 3 nM for des-acyl Ghr (BIAcore end point analysis, data not shown). A more accurate determination of antibody affinity/specificity was undertaken by BIAcore due to the instrument's ability to monitor real time kinetics as well as its sensitivity to low vs high affinity antibodies. JG4, JG2, and JG3 had relative Kd's of 77.6 pM, 12.3 nM, and 26.9 nM for acyl-ghrelin, confirming mAb JG4 to have the highest affinity to the peptide. Kinetic analysis through BIAcore provided kinetic association and dissociation constants. JG4 and JG2 bind full-length acyl-ghrelin fitting a 1:1 binding with mass transfer model and a 1:1 Langmuir binding model. JG3 however, has relatively slow kon/koff rates and as such fits a two state reaction model in which a conformational change of the peptide is hypothesized to occur during the association phase.

To examine epitope fidelity, the truncated peptides Ghr 1-5 and Ghr 1-10 were tested. As anticipated, only mAb JG4 presented binding for these N-terminal fragments with relative affinities of 1.01 nM and 136.0 pM respectively. These BIAcore analyses further validate JG4's recognition of the octanoylated serine-3 residue with high specificity to the N-terminus of acyl-ghrelin.

Through simple epitope mapping it was determined that JG4 and JG2 recognize distinct binding epitopes on ghrelin, whereas JG3 must recognize a “conformational epitope” that may overlap with the recognition sequence for JG2 and JG4. Specifically, initial binding of JG3 to ghrelin did not exclude the subsequent binding of JG2 or JG4 mAbs to the ghrelin/JG3 complex. However, initial binding of either JG2 or JG4 to ghrelin excluded the subsequent binding of JG3 to the JG2- or JG4-ghrelin complex. These data suggest that, upon JG3 binding to ghrelin, a conformational change occurs, perhaps in which the naturally extended secondary structure of ghrelin folds into a more stable loop/supercoil. Conversely, the initial binding of JG2 or JG4 may prevent the linear ghrelin from folding, which is hypothesized to be crucial for forming a stable complex with JG3.

### Example 2

### JG3:JG4 Doublet Uniquely Alters Whole-Body Metabolism

It was also found that JG3:JG4 doublet uniquely alters whole-body metabolism in fasted and refeeding mice. Due to the high binding affinity of JG4 1C4 to acyl-ghrelin, we began calorimetry experiments by injecting mice subcutaneously with only mAb JG4 at a dose of 15 mg/kg. Initial calorimetric and food intake studies demonstrated no significant differences between monoclonal antibody JG4 1C4 and the nicotine control antibody NIC-1 9D9 in male C57BL/6J mice for any of the whole-body metabolism measures (heat, VO₂, VCO₂, and respiratory exchange ratio [RER]) at any time point of the fast or refeeding stages (data not shown). JG4 1C4 also did not alter cumulative food intake after the 24 h fast at any time point through 6 h (data not shown). Based on our positive active immunization findings, we were led to ask whether an oligoclonal antibody response might better enable acyl-ghrelin sequestration, as has been observed for other systems. Thus, monoclonal antibodies were further examined as doublets JG2:JG4, JG3:JG4, and JG2:JG3 at 7.5 mg/kg per antibody.

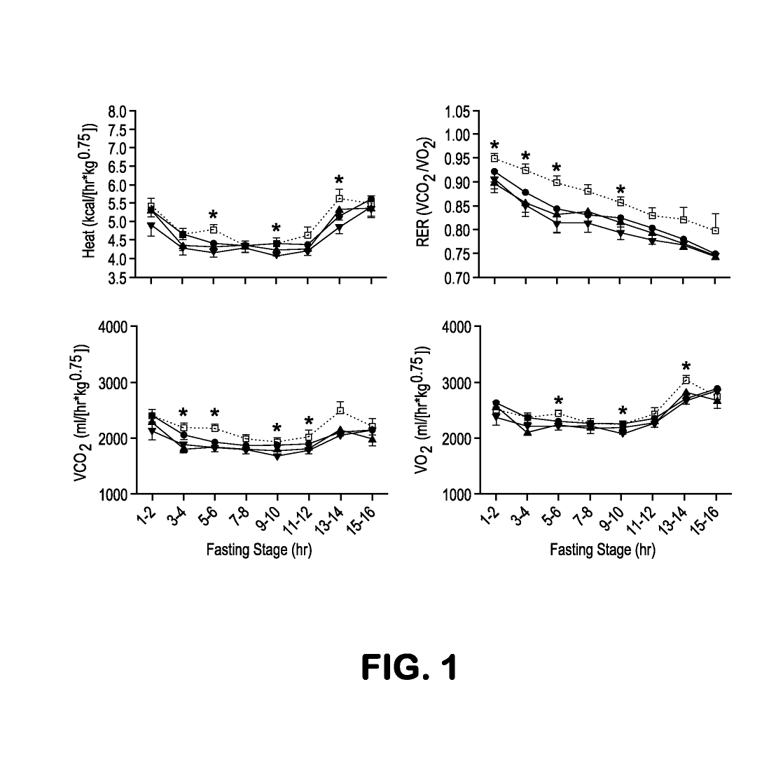

Main effects of doublet indicated that the ghrelin doublet cocktails elicited effects on energy expenditure (heat; F(3,30)=3.570, p<0.025; VCO₂; F(3, 30)=4.003, p<0.016; VO₂; F(3, 30)=2.431, p<0.085); and respiratory exchange ratio (RER; F(3, 30)=3.642, p<0.024). Pair-wise comparisons showed that subjects injected with doublet JG3:JG4 demonstrated significantly higher heat dissipation (kcal/h·kg0.75) (heat; F(1,14)=8.565, p<0.011), with increased CO₂ emission (VCO₂; F(1,14)=6.898, p<0.020) and O₂ consumption (VO₂; F(1,14)=4.511, p<0.052), as well as greater respiratory exchange ratio (RER; F(1,14)=7.511, p<0.016) during the first 14 h of the fast as compared to nicotine control mAb subjects (FIG. 1). In contrast, mice treated with JG2:JG4 showed a main effect only on heat dissipation (heat; F(1,14)=6.756, p<0.021), however, pairwise analysis of individual time bins showed no significance. The JG2:JG3 doublet did not differ from control mAb-treated mice within any time-bin of the fast on the metabolic measures.

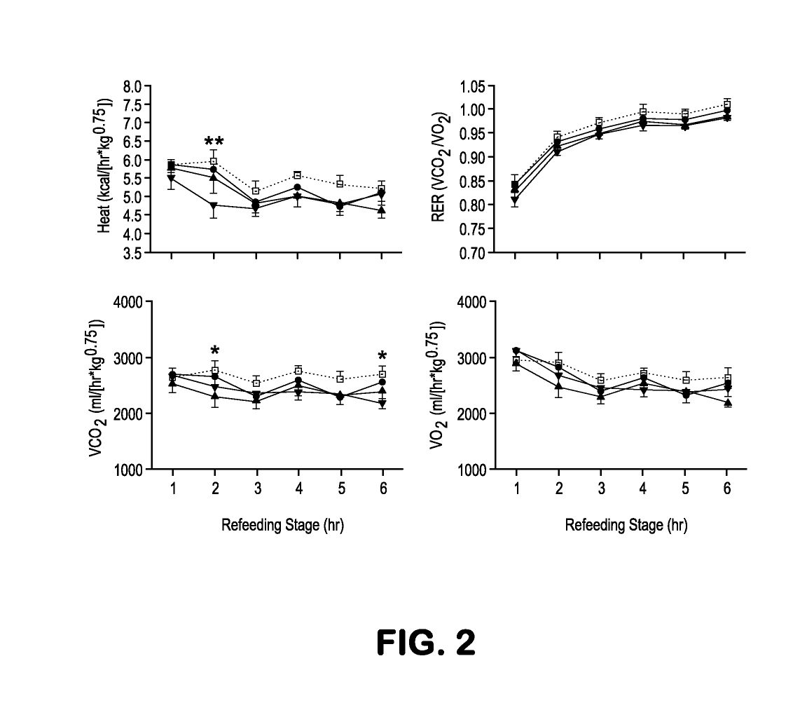

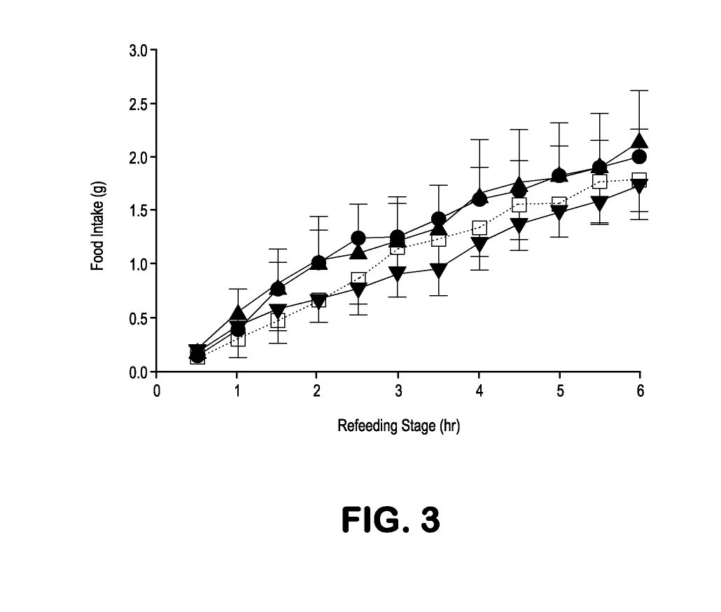

During the refeeding stage, mice injected with doublet JG3:JG4 demonstrated significant increase in heat dissipation during hour 2 as well as increased CO2 emission during hours 2 and 6 (FIG. 2, heat; F(1, 14)=8.869, p<0.010; VCO2; F(1, 14)=6.601, p<0.022), suggesting the continuation of relative increases in energy expenditure even during energy repletion. During refeeding, a trend was observed in VO2 consumption across groups (VO2; F(3, 30)=2.498, p<0.079), whereas, respiratory exchange ratio no longer differed reliably between groups (RER; F(3, 30)=1.830, p<0.163). Caloric intake across the 6 h period was not significantly different between any of the doublet groups vs the nicotine control group (FIG. 3; intake; F(3, 29)=0.387, p<0.763).

### Example 3

### mAb Triplet Alters Whole-Body Metabolism and Reduces Caloric Intake

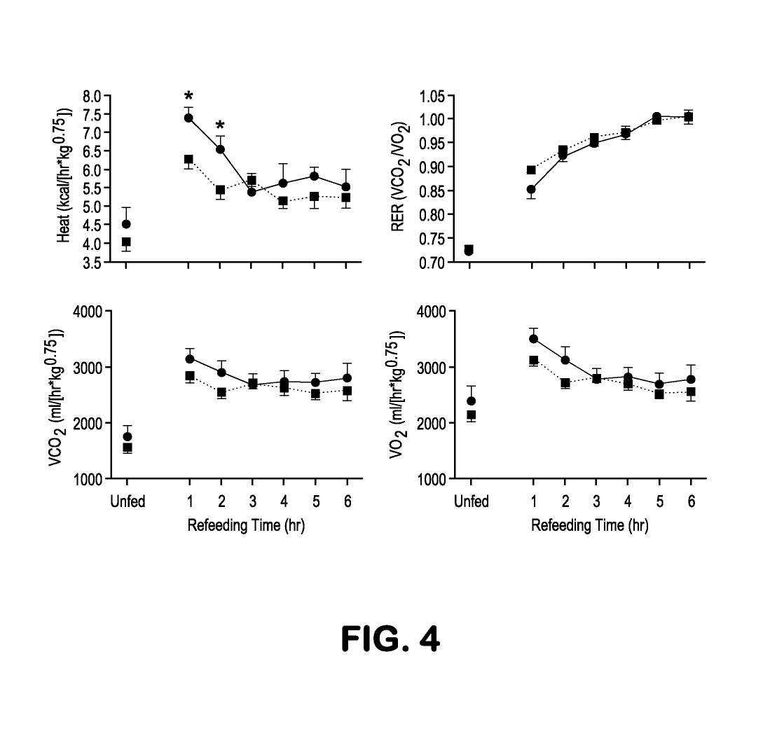

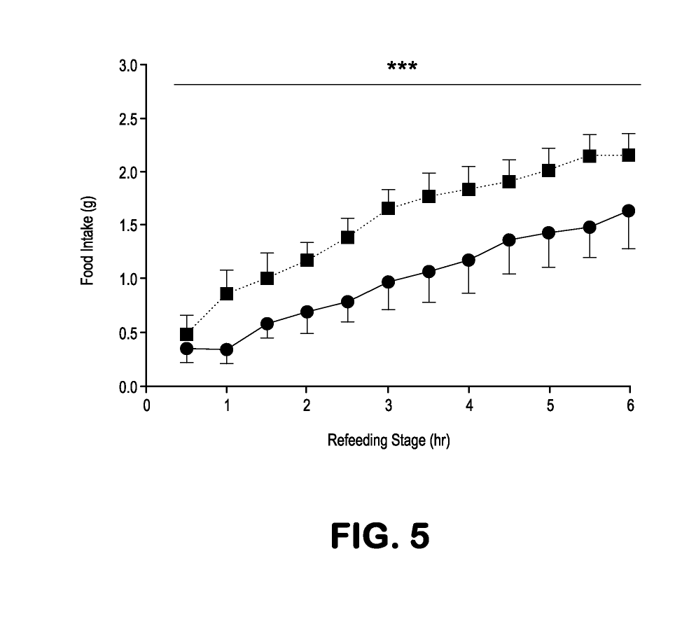

When mAbs were administered as a “cocktail” combination of all three antibodies JG4, JG3, and JG2 (but still at a total dose of 15 mg/kg), significant increases in heat dissipation and O2 consumption were noted during hours one and two of the refeeding stage (FIG. 4; heat hour 1; F(1,8)=7.523, p<0.025; heat hour 2; F(1, 8)=6.319, p<0.036; VO2 hour 1; F(1, 8)=8.189, p<0.021; VO2 hour 2; F(1,8)=8.136, p<0.021). The mice administered the cocktail also demonstrated significantly less cumulative caloric intake during the first 6 h of the refeeding stage as compared to the nicotine control mice (FIG. 5; intake; F(1, 15)=1.751, p<0.0001). Significant reductions in intake were evident as early as 30 min following the return of food to the chambers.

### Example 4

### Materials and Methods

Synthesis and Screening of Anti-Ghrelin Antibodies. All haptens were prepared on a 1.0 mmol scale using solid-phase peptide synthesis. The first hapten was composed of the first 10 amino acids of ghrelin (N-terminus), with an additional cysteine residue appended at the C-terminus. The immunogenic carrier protein keyhole limpet hemocyanin (KLH) was conjugated to the 10-mer using a sulfosuccini-midyl-4-[N-maleimidomethyl]cyclohexane-1-carboxylate (SMCC) linker at the C-terminal cysteine. The antibody generated from this hapten was labeled JG4 1C4. Monoclonal antibody JG3 8H11 was elicited against full-length ghrelin-KLH, but wherein the octanoylated ester was abbreviated to a butyryl ester due to insolubility issues. JG2 7B4 monoclonal antibody was elicited against ghrelin amino acids 13-28-KLH. All the haptens and substrates were prepared as C-terminal amides. The subsequent monoclonal antibodies were purified from ascites using ion-exchange and protein G affinity chromatography.

Binding Analysis by BIAcore. A BIAcore 3000 instrument (BIAcore, Uppsala, Sweden) was utilized to determine directly whether the mAbs interacted with full-length acyl-ghrelin and des-acyl ghrelin peptides or ghrelin fragments (Ghr 1-5 and 1-10, Peptides International, Inc.). Each tested mAb was immobilized onto a CM3 or CM5 chip using the NHS/EDC coupling method, as well as an in-line reference flow cell coupled with an unrelated mAb (NIC-1 9D9). All measurements were conducted in HEPES buffer (pH 7.6) containing 0.15 M NaCl, 3 mM EDTA, and 0.005% surfactant P20 (HBS-EP) running buffer at 25° C. with a flow rate of 30 μL/min. Acyl, des-acyl ghrelin, or ghrelin fragments were prepared in running buffer at various concentrations ranging from 1 nM to 50 μM and then injected over the mAb-immobilized flow cells. The binding response observed 2.5 min after the end-of-injection was used to define the binding capacity for each mAb against different concentrations of ghrelin peptides. A response ten times higher than the standard deviation of buffer injections was considered “positive” binding.

Kinetic Analysis by BIAcore. To determine the kinetic constants of each defined mAb against acyl-ghrelin or ghrelin fragments, mAbs immobilized onto a CM3 chip were targeted at 3000 RU (for acyl-ghrelin), or a CM5 chip at 6,000 RU (for Ghr 1-10) and 10,000 RU (for Ghr 1-5) respectively, in order to acquire similar Rmax values across samples. An in-line reference flow cell was immobilized with an unrelated nicotine mAb (NIC-1 9D9) at the same level as the ghrelin mAbs. Ghrelin was diluted in running buffer so as to obtain a series of concentrations ranging from 0.2 nM to 4 μM. At least 5 different concentrations of ghrelin were injected into the flow cell for repeated analysis with double references, and the interaction between soluble ghrelin and mAb was recorded on a sensorgram. The kinetic data were evaluated via fitting the experimental data with BIAevaluation software (v. 4.1) using an appropriate kinetic model. Kinetic constants, including the association and dissociation equilibrium rate constants (Ka and Kd) and association and dissociation rate constants (kon and koff), were calculated separately for each mAb. All BIAcore kinetic analyses were double-referenced, i.e., with a reference flow cell and blank buffer run.

Epitope Mapping by BIAcore. To reveal the steric relationships between antigenic sites on acyl-ghrelin that were potentially recognized by the defined mAbs, a pairwise epitope mapping experiment was performed using surface plasmon resonance (SPR) technology. For this, each defined mAb was immobilized onto a CM5 chip via NHS/EDC amine-coupling, followed by the injection of a saturating concentration (10 μM) of acyl-ghrelin. Once the surface was saturated, a secondary mAb was injected, and binding activity was observed in real-time via the sensorgram. This procedure was repeated for all mAbs with all possible binary combinations undertaken, and the binding signal for each pair of mAbs was recorded in a reactivity pattern matrix for further analysis. All BIAcore epitope mapping analyses were double-referenced to a reference flow cell (with the unrelated antinicotine antibody) and blank cell.

Animal Protocols and Antibody Administration. All mouse studies adhered to the National Institutes of Health Guide for the Care and Use of Laboratory Animals and were approved by the Institutional Animal Care and Use Committee of The Scripps Research Institute. Twelve adult male C57BL/6J mice (25.0-28.3 g) were purchased from Charles River Laboratories (Frederick, Md.) for each of 4 replicate experiments (n=48 total). Mice were housed in single cages in a temperature controlled vivarium (60% humidity, 22° C.) with a 12 h light/12 h dark cycle (6:30 am to 6:30 pm) with ad libitum access to water and standard pelleted chow diet (LM-485 Diet 7012; Harlan Teklad, Madison, Wis.) for 2-3 weeks prior to antibody injection. Antibodies were prepared in phosphate buffered saline (pH 7.4) and injected subcutaneously in 130 μL volumes to obtain final total administered antibody doses of 15 mg/kg (5 mg/kg per mAb for triplet injection, 7.5 mg/kg each for doublets). mAbs were injected five days prior to calorimetric experiments to allow adequate distribution in vivo.

Metabolic and Food Intake. Subjects were singly housed and acclimated (>72 h) to indirect calorimetry chambers where O₂ consumption, CO₂ emission, and heat dissipation were measured using a computer-controlled, open-circuit system (Oxymax System) that was part of an integrated Comprehensive Lab Animal Monitoring System (Columbus Instruments, Columbus, QH). The Plexiglas test chambers (20×10×12.5 cm) had a stainless steel elevated wire floor and were equipped with a sipper tube delivering water and a food tray connected to a balance. Room air was passed through chambers at a flow rate of 0.5 L/min. Exhaust air from each chamber was sampled at 20 min intervals for 30 s. Sample air was sequentially passed through O₂ and CO₂ sensors (Columbus Instruments) for determination of O₂ and CO₂ content, from which measures of oxygen consumption (VO₂) and carbon dioxide production (VCO₂) were estimated. Outdoor air reference values were sampled after every four measurements. Gas sensors were regularly calibrated with primary gas standards containing known concentrations of O₂, CO₂, and N2 (Airgas Puritan Medical, Ontario, Calif.). Respiratory exchange ratio (RER) was calculated as the ratio of carbon dioxide production (VCO2) to oxygen consumption (VO2). Energy expenditure measures (VO2, VCO2, and heat formation [(3.815+1.232×RER)×VO2 (in liters)] were corrected for estimated effective metabolic mass per Kleiber's power function. Data were recorded under ambient room temperature (24-26° C.). During acclimation, powdered chow and water were available ad libitum. Subjects were then subjected to a 24 h fast beginning from the onset of the light cycle, during which water remained available. At the onset of the next light cycle, mice were allowed to refeed for 6 h, during which food intake was automatically measured by a computer-monitored scale bearing the food tray (0.1 g precision). Data were graphed using Prism software (GraphPad Software Inc., San Diego, Calif.). All values were plotted as the mean±standard error of the mean (SEM).

Statistical Analysis. Data were analyzed by two-way analysis of covariance (ANOVA), with hapten group as a between-subject factor (e.g., vehicle vs doublet AB vs doublet AC vs doublet BC), time as a repeated measure (where applicable), and cohort as a covariate. To ensure reproducibility of findings, subjects were run in 4 independent cohorts per experiment (2×), balanced for treatment group. Significant omnibus tests (p<0.05) were further interpreted by simple effects analysis and by ANCOVAS comparing individual hapten groups to the vehicle condition. Results are expressed as mean±SEM. The statistical package used was Systat 12.0 (SPSS, Chicago, Ill.).

All publications, databases, GenBank sequences, patents, and patent applications cited in this specification are herein incorporated by reference as if each was specifically and individually indicated to be incorporated by reference.

What is claimed is:

1. An antibody comprising heavy and light chains, each having a complementarity determining regions (CDRs) of the antibody produced by the hybridoma cell line selected from the group consisting of ATCC™ deposit number PTA-120176, PTA-120174, and PTA-120175 or each having at least 95% amino acid sequence homology to the CDRs of the antibody produced by the hybridoma cell line selected from the group consisting of ATCC™ deposit number PTA-120176, PTA-120174, and PTA-120175.

2. The antibody or antigen-binding molecule of claim 1 which is a scFv fragment, an Fv fragment, an Fd fragment, an Fab fragment or an F(ab′)₂ fragment.

3. An isolated or recombinant polynucleotide which encodes a polypeptide comprising the variable region of the heavy chain or the variable region of the light chain of the antibody of claim 1.

4. A hybrid cell line which produces a monoclonal antibody, the monoclonal antibody being specifically reactive with ghrelin and has the specificity of the antibody produced by a hybridoma cell line selected from the group consisting of ATCC™ deposit number PTA-120176, PTA-120174, and PTA-120175.

5. The cell line of claim 4, wherein the antibody is a catalytic antibody capable of degrading ghrelin.

6. The cell line of claim 4, which has a hybridoma cell line selected from the group consisting of ATCC™ deposit number PTA-120176, PTA-120174, and PTA-120175.

7. A pharmaceutical composition comprising a therapeutically effective amount of the antibody of claim 1 and a pharmaceutically acceptable vehicle.

8. The pharmaceutical composition of claim 7, wherein the antibody is produced by the hybridoma cell line selected from the group consisting of ATCC™ deposit number PTA-120176, PTA-120174, and PTA-120175.

9. The pharmaceutical composition of claim 7, wherein the antibody is a scFv fragment, an Fv fragment, an Fd fragment, an Fab fragment or an F(ab′)₂ fragment.

10. A method of reducing weight or slowing weight gain in a subject, comprising administering to the subject a pharmaceutical composition comprising an antibody comprising heavy and light chains, each having a complementarity determining regions (CDRs) of the antibody produced by the hybridoma cell line selected from the group consisting of ATCC™ deposit number PTA-120176, PTA-120174, and PTA-120175 or each having at least 95% amino acid sequence homology to the CDRs of the antibody produced by the hybridoma cell line selected from the group consisting of ATCC™ deposit number PTA-120176, PTA-120174, and PTA-120175, thereby reducing weight or slowing weight gain in the subject.

11. A method of treating obesity in a subject, comprising administering to the subject a pharmaceutical composition comprising An antibody comprising heavy and light chains, each having a complementarity determining regions (CDRs) of the antibody produced by the hybridoma cell line selected from the group consisting of ATCC™ deposit number PTA-120176, PTA-120174, and PTA-120175 or each having at least 95% amino acid sequence homology to the CDRs of the antibody produced by the hybridoma cell line selected from the group consisting of ATCC™ deposit number PTA-120176, PTA-120174, and PTA-120175, thereby treating obesity in the subject.
